(19) United States
(12) Patent Application Publication
(10) Pub. No.: US 2020/0306518 A1
(43) Pub. Date: Oct. 1, 2020

(54) SYSTEM AND METHOD FOR APPLYING MICRONEEDLES

(71) Applicant: UPRAX MICROSOLUTIONS B.V., Delft (NL)

(72) Inventors: Koen VAN DER MAADEN, Leiden (NL); Bernardus Hendrikus VAN OORSCHOT, 's-Gravenhage (NL)

(21) Appl. No.: 16/303,627

(22) PCT Filed: May 22, 2017
(86) PCT No.: PCT/EP2017/062238

§ 371 Date: Nov. 20, 2018

(30) Foreign Application Priority Data
May 20, 2016 (NL) 2016807
May 23, 2016 (EP) 16170775.7

(51) Int. Cl. A61M 37/00

(52) U.S. Cl. CPC A61M 37/0015; A61M 2205/33; A61M 2037/0023

(57) ABSTRACT

A microneedle applicator system comprising an applicator and a controller. The applicator comprises an electromechanical actuator arranged in an outer housing to apply a microneedle module via a supporting body onto skin. The electromechanical actuator is arranged to displace the supporting body between a first position inside the outer housing, and a second position substantially in line with an opening in the housing or outside the housing. The controller is arranged to control a velocity of the actuator and/or a pressure force of the actuator by regulating the power that is supplied to the actuator. The controller comprises switching circuitry for switching the power supplied to the actuator on and off, and an integrated circuit arranged to generate a pulse width modulated signal and to control the switching circuitry by setting or adjusting a duty cycle of the pulse width modulated signal.

### FIELD OF THE INVENTION

The invention relates to a microneedle applicator system, and to a method of applying microneedles onto skin.

### BACKGROUND ART

Dermal drug delivery has several advantages over conventional drug delivery methods, especially for drugs with a low bioavailability due to absorption in the gastrointestinal tract and/or due to first pass metabolism. Dermal drug delivery is also advantageous for therapeutic and prophylactic vaccination because the skin is highly immune responsive owing to the large numbers of Langerhans cells and dendritic cells. Furthermore, the skin has a large surface area that is available for drug delivery and is an easily accessible route of administration. However, only about 20 active pharmaceutical ingredients have been formulated in approximately 40 pharmaceutical products for dermal application due to the stratum corneum, which is generally 10-20 μm thick in human and has a low permeability for most drug molecules.

To overcome the stratum corneum several dermal drug delivery systems and devices have been developed, including microarrays or microneedles, which are devices that contain one or more needles with micron-sized dimensions. Several types of microneedle technologies have been developed for dermal and transdermal drug delivery and/or sampling of biological samples from the skin.

Patent publication WO2014092566A1 discloses a method of producing and applying a hollow or solid microneedle made from fused silica capillaries for dermal drug delivery and sampling of biological fluids. WO2009072830A2 describes an array of hollow microneedles for drug delivery and sampling of biological fluids via the skin.

U.S. Pat. No. 8,414,548B2 describes a method of producing solid microneedle arrays and a device for the application of microneedle arrays onto skin.

US20140142541A1 discloses the production and application of an array of dissolving microneedles that contain nanomaterials, which dissolve upon piercing of the skin within five minutes. US20120265145A1 describes dissolving microneedles that dissolve by hydrolysis once the microneedles penetrate skin.

US2010280457 discloses a method to coat a microneedle or a microneedle array by applying a high viscosity coating onto the surface of the microneedle. WO2013036115A1 describes a method to coat a microneedle or a microneedle array with a drug by modifying the microneedle surface with nanolayers to minimize the reduction of sharpness upon coating of microneedles.

A large variety of microneedles or microneedle arrays have been fabricated with many different geometries. Examples are microneedles that vary in length, sharpness, shape (conical, cylindrical, pyramid, etc.), number of needles per array, density, and these microneedles may be hollow, solid or porous. Microneedles or microneedle arrays can be made of several different materials, such as glass, silicon, stainless steel, titanium, sugar, or polymers. Also, microneedles may be coated with a drug, for example by using highly viscous coating solutions that adhere to the microneedle, thereby leaving thick layers of coatings on the microneedle surface, resulting in a decreased microneedle sharpness and/or increased surface roughness.

The type of microneedle, microneedle geometry and the material from which the microneedles are made can influence their skin penetrating ability. Furthermore, coating of microneedles may result in a decreased sharpness of the microneedles and thereby result in a decreased penetration efficiency of skin by these microneedles.

The common factor for all types of microneedles is that they must pierce the stratum corneum in order to deliver a drug into the skin or obtain a biological sample from the skin. Furthermore, for drug delivery purposes microneedles must penetrate the skin effectively and reproducibly when applied onto skin. Therefore, each type of microneedle or microneedle array should be applied onto skin with its optimal application parameters.

The microneedle material, geometry, and/or coating influence the microneedle strength, sharpness, and/or surface roughness, and thereby its skin penetration ability, and thereby its optimal application parameters. Thus, for each type of microneedle device, and/or alteration of the geometry and/or coating of a microneedle device requires investigation and/or reinvestigation of the optimal application parameters.

Several microneedle applicators have been developed. These applicators are generally impact applicators, which are devices that propel microneedles with a certain velocity towards the skin, or applicators that deliver a given amount of pressure via the microneedles onto skin.

US 20050261631 discloses a microneedle applicator apparatus which uses springs to apply a drug delivery system onto the skin. US20070027427 discloses a method to apply a drug delivery system onto the skin by using multiple springs in order to first apply a drug delivery system onto the skin and subsequently retract the drug delivery system into the applicator. US20040215151 also discloses an applicator that uses springs to move a drug delivery system onto and from the skin and shrouds the needle after use. These spring-based applicators that are used to apply microneedle modules onto skin by a momentum offer no little control over application parameters.

U.S. Pat. No. 6,611,707B1 discloses an applicator to apply microneedles through pressure by manual application. The pressure is obtained by pressing a reservoir by hand to deliver a drug formulation into the skin. Further, U.S. Ser. No. 10/238,958 discloses an applicator which uses hand pressure to drive a plunger which forces a drug formulation through the microneedle into the skin. These types of applicators to apply microneedles by hand pressure onto the skin give little or no control over the application force and other application parameters.

WO2009077859 does describe a microneedle injecting apparatus which is disclosed to apply drug delivery systems and/or sampling devices onto skin via an electrically-controlled actuator by a momentum. However, the actuator control is mainly via charging and discharging of capacitors, which limits the flexibility and applicability to accurately control the applied momentum and the application time.

### SUMMARY OF THE INVENTION

It is an object of the invention to provide a microneedle applicator system which solves at least one of the above-mentioned problems of the state of the art.

A first aspect of the invention provides a microneedle applicator system comprising an applicator and a controller. The applicator comprises an outer housing and an electromechanical actuator arranged in the outer housing and arranged to apply a microneedle module via a supporting body onto skin. The electromechanical actuator being arranged to displace the supporting body between a first position in which the supporting body is positioned inside the housing, and a second position in which the supporting body is substantially in line with an opening in the housing or outside the housing.

The controller is arranged to control a velocity of the actuator and/or a pressure force of the actuator by regulating the power that is supplied to the actuator. The controller comprises a switching circuitry for switching the power supplied to the actuator on and off, and an integrated circuit arranged to generate a pulse width modulated signal and to control the switching circuitry by setting or adjusting a duty cycle of the pulse width modulated signal.

Using a pulse width modulated signal to control the switching circuitry, and thus the actuator, provides for a very flexible microneedle applicator system which gives a user more precise control. By setting or adjusting a duty cycle of the pulse width modulated, the velocity and/or pressure force of the actuator can be set accurately or adjusted to find optimal settings during experimental tests.

The microneedle module may be a drug delivery system and/or sampling device. The microneedle module may comprise a single microneedle or multiple microneedles, possibly arranged in an array. The supporting body may be a supporting plateau, or any other suitable body arranged to support or attach a needle, a needle array, or a module with needles.

It is noted that the applicator and the controller may be combined into one device, but alternatively they are two or more separate devices communicating with each other.

Optionally, the actuator comprises a voice coil arranged to move the supporting body from a first position to a second position within or outside the applicator. Using a voice coil provides for the possibility to electronically control over the first position and second position. Besides, the velocity and acceleration can be accurately controlled.

Optionally, the system further comprises a user interface, and wherein the controller is arranged to operate the applicator in at least a first mode and a second mode, wherein in the first mode the applicator is retracted from an extended position to a retracted position as soon as a user selectable pressure is detected, and in the second mode the applicator is extended from a retracted position into an extended position with a user selectable momentum. This provides for a very flexible system for R&D purposes in order to find the optimum application parameters for certain microneedle types and certain skin types.

Optionally, the applicator comprises an inner housing adjustably arranged inside the outer housing wherein by adjusting the position of the inner housing along a main axis of the actuator, the second position of the supporting body can be set between a zero-position in which the supporting body is substantially in line with the opening and a protruded position in which the supporting body is outside the outer housing. In this way, the user can choose to use the applicator to apply microneedles via a pressure or impact. Furthermore, if microneedles are applied by impact, the position of the supporting body can be adjusted in such a way that only microneedles (and not the base/back plate) protrude the outer housing.

Optionally, the applicator comprises piezoelectric element arranged onto the inner housing, wherein the controller is arranged to determine a maximum extended position of the supporting body by measuring a signal from the piezoelectric element and compare it to a threshold voltage, and if the measured signal exceeds the threshold voltage, retract the supporting body into the outer housing after a predetermined period of time.

Optionally, the supporting body is adjustably arranged onto the actuator wherein by adjusting the position of the supporting body along a main axis of the actuator, the second position of the supporting body can be set between a zero-position in which the supporting body is substantially in line with the opening and a protruded position in which the supporting body is outside the outer housing.

Optionally, the actuator is controlled in such a way that:

I. at first, the position of the actuator is set in such a way that the supporting body protrudes out of the applicator; and

II. subsequently, after the applicator is applied to the skin by a user pressing the microneedle module via the supporting body onto skin the supporting body is partially protruded when the holding force is reached, the supporting body remains partially protruded for a predetermined period of time, for holding the microneedle module onto skin for the predetermined period of time; and

III. subsequently, the supporting body is retracted into the applicator when a predetermined time is reached.

This way of controlling gives multiple advantages. The pressure to apply microneedles is very controlled. Users have time to apply microneedles with a certain pressure. Slower/older users can have more time for the application of microneedles. Also, a longer application time can result in better (e.g., higher/more reproducible) application and/or drug delivery. And finally, (if microneedles are onto supporting body) the microneedles are automatically retracted, not reaching potential biohazardous waste.

Optionally, A system according to any one of the claims 1-6, wherein the actuator is controlled in such a way that:

I. at first, the position of the actuator is set in such a way that the supporting body is inside the applicator; and

II. subsequently, after the applicator is placed onto skin by a user, the actuator moves the supporting body towards the skin until the supporting body reaches the skin, wherein the supporting body is pressing the microneedle module onto skin until a predetermined pressure is reached; and

III. subsequently, the supporting body remains at its position holding the microneedle module onto skin for a predetermined period of time; and

IV. subsequently, the supporting body is retracted into the applicator.

Optionally, the actuator is controlled in such a way that:

I. at first, the supporting body is in a retracted state in the applicator; and

II. subsequently, after the applicator is placed onto the skin by a user, the supporting body is propelled towards a zero position; and

III. subsequently, the supporting body is retained in the zero-position for a predetermined period of time; and

IV. subsequently, the supporting body is retracted into the applicator.

This way of controlling gives multiple advantages. The impact to apply microneedles is very controlled. Users can press a button to apply microneedles via impact onto skin. Furthermore, a longer application time can result in better (e.g., higher/more reproducible) application and/or drug delivery. If the microneedles are onto the supporting body, the microneedles are automatically retracted not reaching potential biohazardous waste.

Optionally, the supporting body is propelled multiple times with a predetermined application frequency towards the zero position. During tests using coated microneedles propelled onto skin multiple times, the required time to immunize was reduced from 90 minutes (impact insertion (50 cm/sec) and holding the microneedles onto skin for 90 minutes) to 10 seconds (10 repeated impact insertions of 50 cm/sec at a frequency of 1 Hz). Furthermore, repeated insertion can lead to better (i.e. more effective, reproducible and/or faster application of microneedles.

Optionally, the application frequency is adjustable between 0.01 mHz and 50 Hz. Changing the frequency may lead to further improved release/better application.

Optionally, the controller is arranged to adjust the number of insertions between 2 and 106, wherein one insertion cycle is the time that the actuator is in the zero position plus the time that the actuator is (partially) retracted into the outer housing of the applicator.

Optionally, the controller is arranged to adjust a time and/or ratio that the supporting body is in the zero position and (partially) retracted in the outer housing of the applicator within one insertion cycle.

Optionally, the controller is arranged to read a value indicative of an amount of pressure force onto the supporting body or indicative of a momentum of the actuator from a calibration table. In this way it can be determined what PWM to use for a specific velocity or pressure. In the device the required PWM may be calculated for a specific velocity or pressure from a calibration curve.

Optionally, the system comprises a pressure sensor, at least in use between the skin and the actuator, and arranged to sense an applied pressure on the supporting body, wherein the controller is arranged to control the power of the applicator using feedback from the pressure sensor.

Optionally, the system comprises a velocity sensor arranged to measure a velocity of the actuator, wherein the controller is arranged to calculate or receive the measured velocity and to control an average velocity of the actuator between a retracted position and a zero position or the velocity at a zero position using feedback of the velocity sensor. In this way the exact velocity of each insertion can be determined, which is useful when using feedback in the system, or when doing research for finding the optimal parameters.

Optionally, the sensor is a linear encoder or a piezoelectric element.

Optionally, the controller is arranged to execute a software program so as to digitally adjust the time that the supporting body is in the first and/or second position and/or delivers a required force to retain a drug delivery and/or sampling device for a pre-installed application time onto the skin.

Optionally, the application time is between 1 and 999 milliseconds.

Optionally, the application time is between 1 and 50 milliseconds.

A second aspect of the invention provides a controller for use in a system as described above.

A third aspect of the invention provides an applicator for use in a system as described above.

A fourth aspect of the invention provides a method of applying microneedles onto skin. The method comprises:
apply a microneedle module via a supporting body onto skin, by using an applicator comprising an outer housing and an actuator;
displace the supporting body between a first position in which the supporting body is positioned inside the housing, and a second position in which the supporting body is substantially in line with an opening in the outer housing or outside the outer housing;
electronically control the velocity and/or the pressure force of the applicator by supplying appropriate power to the actuator using a pulse width modulated signal.

### BRIEF DESCRIPTION OF THE DRAWINGS

These and other aspects of the invention are apparent from and will be elucidated with reference to the embodiments described hereinafter. In the drawings:

FIG. 1 schematically shows a system for applying microneedles according to an embodiment;

FIG. 2 shows an exploded view of an applicator according to an embodiment;

FIG. 3A-3C show three positions of a supporting plateau of an applicator with a microneedle module attached;

FIG. 3D-3F show the three positions of a supporting plateau of the applicator without the microneedle module attached;

FIG. 4A-4F show six states of the applicator with a microneedle module attached or placed onto skin, wherein a predetermined force is used;

FIG. 5A-5F show six states of the applicator with a microneedle module attached or placed onto skin, wherein a predetermined momentum is used;

FIG. 6 schematically shows an electrical circuit to control an actuator of the applicator via a pulse width modulation;

FIG. 7 shows a graph of the force of three different solenoids in different applicators as a function of the actuator power;

FIG. 8A shows a graph of the activation time of three different solenoids in different applicators as a function of the actuator power;

FIG. 8B shows a graph of the application velocity of three different solenoids in different applicators as a function of the actuator power.

It should be noted that items which have the same reference numbers in different Figures, have the same structural features and the same functions, or are the same signals. Where the function and/or structure of such an item has been explained, there is no necessity for repeated explanation thereof in the detailed description.

### DETAILED DESCRIPTION OF EMBODIMENTS

The present invention is a device that encompasses an electronic controller and an applicator to digitally control the manner and the force of application of a microneedle module onto skin.

Drug delivery via the skin can have several advantages over drug delivery via other administration routes, because the skin is easily accessible, has a large available surface area, and delivery via this route can be performed on a minimally invasive manner and is potentially pain free. One of the promising systems for dermal drug delivery and taking biological samples from the skin on a minimally invasive and potentially pain-free manner is by the use of a microneedle or a microneedle array.

In this context a “microneedle” is a micron-sized structure that has preferably a length of less than 1 millimeter which is intended to pierce through the stratum corneum into the epidermis and/or dermis. Further, in this context a microneedle module may refer to a dermal delivery and/or a sampling device, which may include needle like structures with lengths up to several millimeters that are intended to pierce through the stratum corneum into the epidermis and/or dermis. A “microneedle array” refers to a structure that contains more than one microneedle.

In this context, “applying” and/or the “application” of microneedles is intended to inject, insert and/or pierce microneedles into the skin.

Microneedles may be used to permeate skin that may be followed by the application of a topical formulation, may be used to extract biological fluids from the skin, and/or deliver a drug into the skin.

In this context, an applicator is a device that is used to apply a microneedle module onto skin. A controller is referred to as a device that controls the applicator.

In the context of this invention, the skin onto which the microneedle module is applied is animal skin, mammalian skin or human skin.

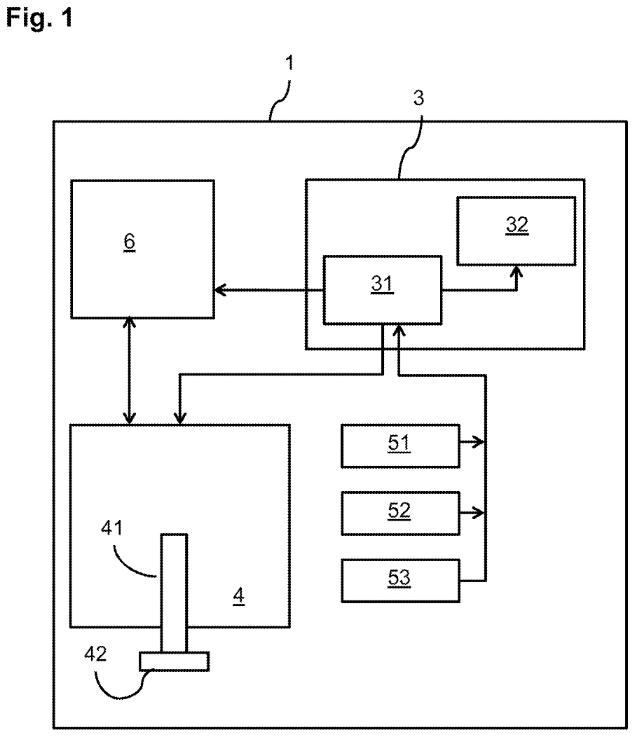

FIG. 1 schematically shows a system for applying microneedles according to an embodiment. The microneedle applicator system 1 comprises a controller 3, an applicator 4 and in this example also a pump 6. Furthermore, the system 1 comprises a temperature sensor 51, a pressure sensor 52 and a position sensor 53. The controller 3 comprises a microcontroller 31 and a user interface 32 which may be a display and/or other user interface equipment, such as buttons and switches.

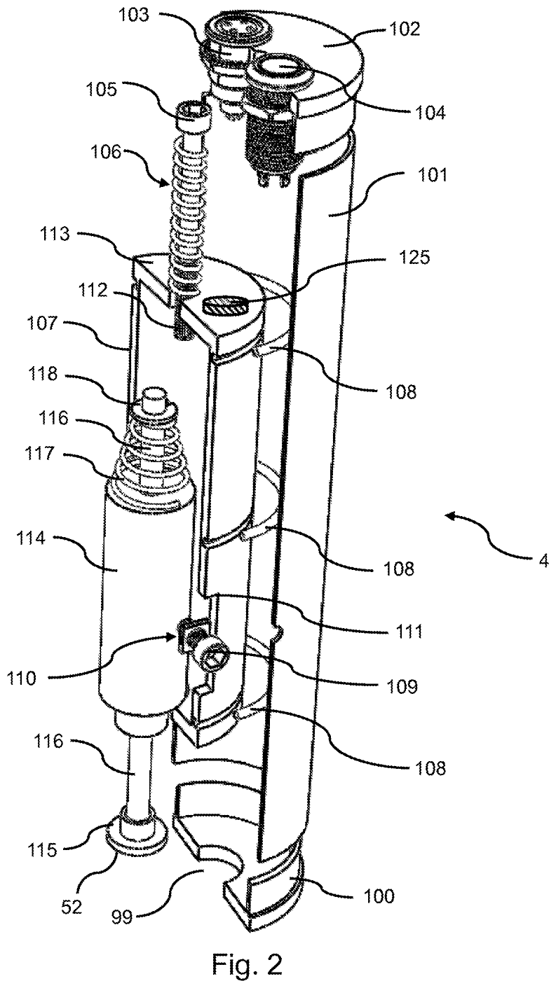

The applicator 4 comprises an actuator 41 and a supporting body 42 which in this example is a supporting plateau 42. The actuator 41 is arranged to displace the supporting plateau 42 between a first position in which the supporting body is positioned inside an outer housing (see also FIG. 2), and a second position in which the supporting body 42 is substantially in line with an opening 99 in the outer housing or outside the outer housing. The controller 3 is arranged to electronically control the applicator 4 wherein the controller 3 is arranged to move the supporting body 42 between a first and a second position by supplying appropriate power to the actuator 41.

The pump 6 may be a syringe pump. The pump 6 may be used to deliver fluids to the needles and thereby to the skin. The pump 6 may also be used to sample fluids coming from the skin.

In an embodiment, the controller 3 is arranged to control the manner of application of a microneedle module onto skin by the applicator 4. This controller-applicator combination may be used to apply microneedle module via an adjustable momentum, via an adjustable pressure, or via a repeated momentum onto skin. In an embodiment, the controller 3 is used to digitally adjust the mode of application and the application parameters using input from a user.

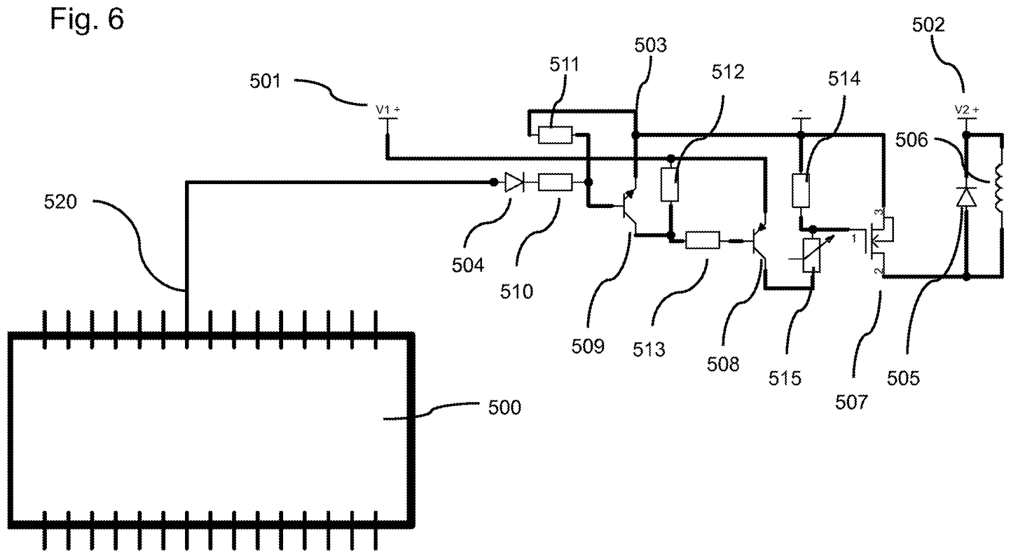

The controller 3 may comprise a microcontroller 500 (see also FIG. 6) which may be programmed to control the applicator to adjust the manner of application and the application parameters to apply microneedle modules onto skin, as exemplified in FIG. 1. These parameters may include the application force, application pressure, application momentum, application velocity, application time, application frequency, number of insertions, and/or time within one application cycle that the supporting plateau is in a zero position or in a retracted position. Further, the controller 3 may simultaneously trigger and/or control an external pump for liquid displacement, either for taking samples or delivering liquids via microneedle modules that are applied onto the skin.

In this context, an “application cycle” is referred to as the time that a supporting plateau is in a zero-position plus the time that a supporting plateau is in a retracted position in a multiple application mode, which is equal to one divided by the application frequency.

Further in this context, the “application frequency” is referred to as the number of application cycles per second, and the “number of insertions” is referred to as the number of application cycles.

In an embodiment of the invention, the application frequency is between 0.01 milli Hertz (mHz) and 50 Hz, preferably between 10 mHz and 25 Hz. Further the number of insertions may vary from two to 10\hat{ }6.

In a further context of this invention, the “zero-/retracted position rate” is referred to as the percentage of time that a supporting plateau is in a zero-position in a complete application cycle.

In the context of this invention, the “application time” is referred to as the time that the supporting plateau is in a zero position or in an extended position.

In an embodiment of this invention, the application time may be adjustable from 1 millisecond to multiple days.

In an embodiment of the invention, the controller generates a pulse width modulation of which the duty cycle is adjustable. Suitable frequencies of a pulse width modulation to drive a solenoid or voice coil are above 1 kilo Hertz (kHz), preferably above 10 kHz. A pulse width modulation is used to switch a transistor to regulate the power from a power source that is send to an actuator in an applicator. The actuator may be a solenoid or a voice coil. The power source may be arranged in the controller or arranged external to the controller.

In the context of this invention the “actuator power” refers to an adjustment of the duty cycle of a pulse width modulation by which a transistor switches a power supply to drive a solenoid or a voice coil.

In a further embodiment of the invention, the transistor that drives the solenoid or voice coil is a power MOSFET, see also 507 in FIG. 6. Examples are IRF520N and IRLZ44N.

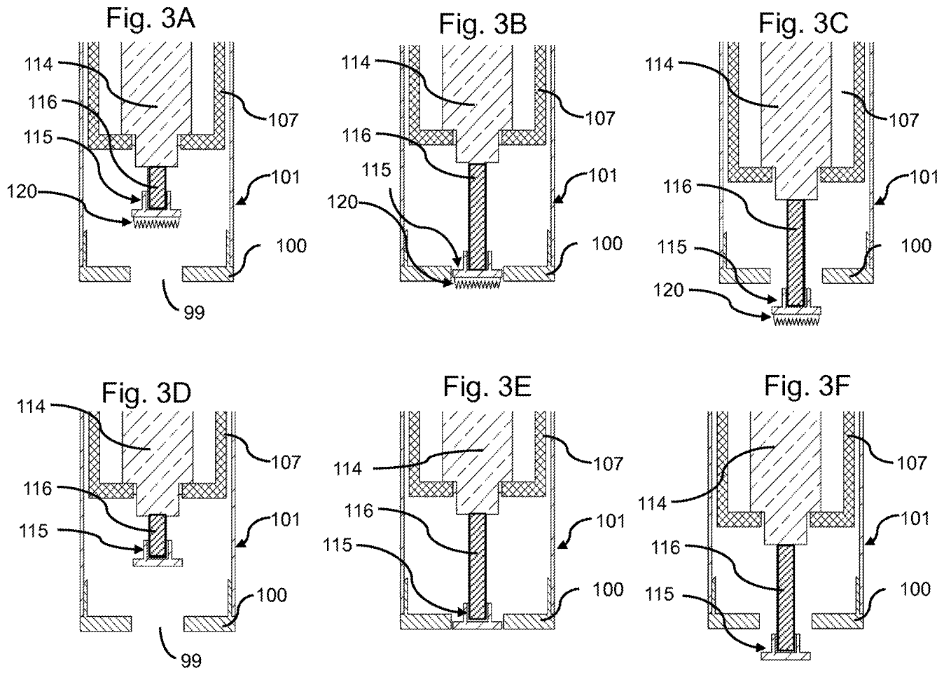

The applicator comprises a supporting body that has an adjustable position in relation to a skin support, and may be in a retracted position, a zero-position or a protruding position. FIG. 3 shows examples of positions of a supporting plateau with and without a microneedle module mounted onto it.

The inner housing of the applicator (FIG. 2) may contain rubber rings between an inner housing and outer applicator shell to vertically align the inner housing.

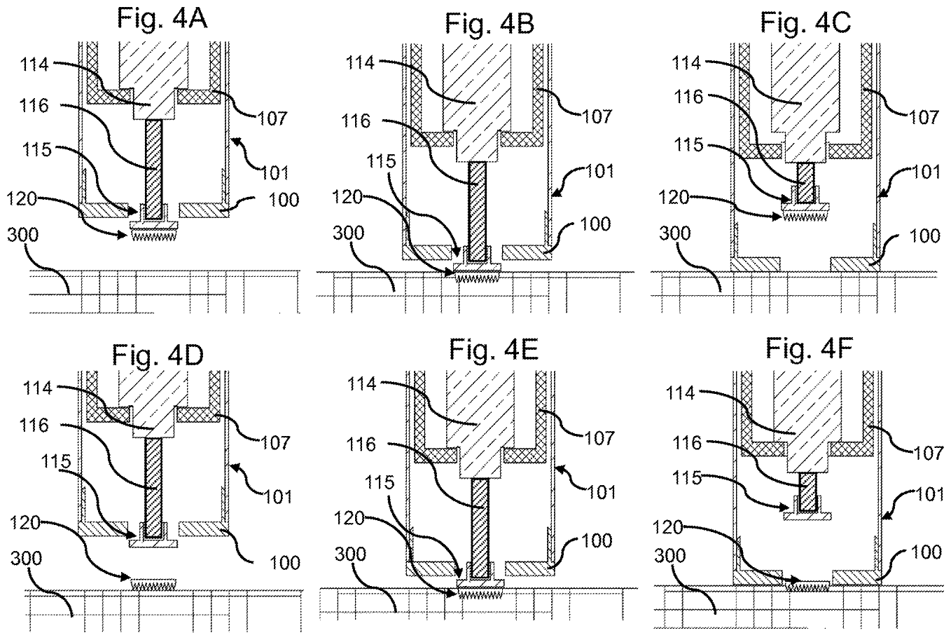

In the context of this invention, the “application pressure” is referred to as the pressure by which an actuator applies a microneedle module via a supporting plateau onto skin. The microneedle module may be mounted onto a supporting plateau or may be placed and/or mounted onto the skin, as schematically represented in FIG. 4.

In a preferred embodiment of this invention, the application pressure that is obtained by a solenoid or a voice coil is digitally-adjustable by adjusting the actuator power.

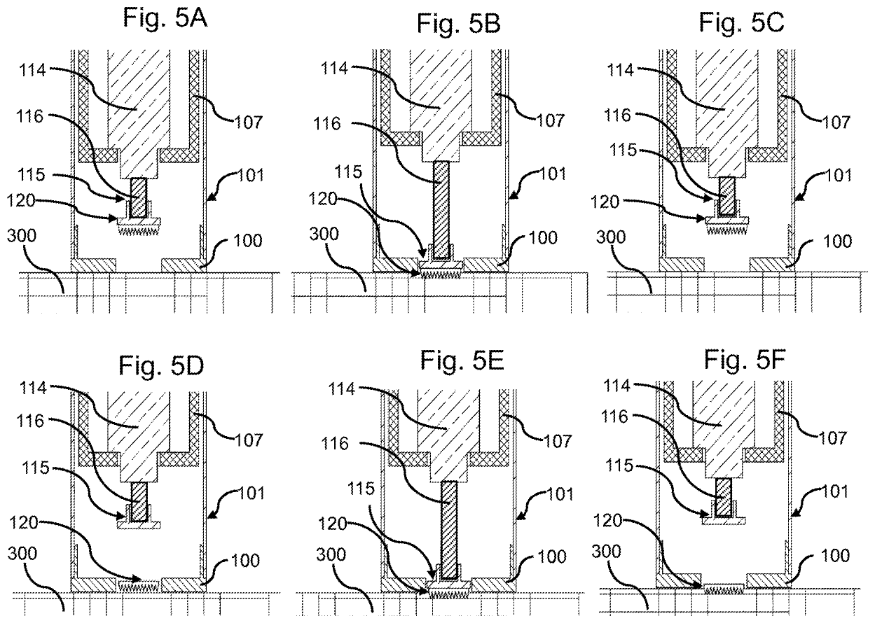

In a context of this invention, the “application momentum” may refer to the momentum by which an actuator in an applicator applies a microneedle module that is mounted onto a supporting plateau onto skin, or may refer to the momentum by which an actuator in an applicator applies a microneedle module that is placed and/or mounted onto skin via a supporting plateau, as shown in FIG. 5.

In a context of this invention, when microneedle modules are applied onto skin by a digitally-adjustable momentum, the supporting plateau propels towards a zero-position towards the skin with a certain velocity, which is referred to as the “application velocity”. The average application velocity for the application of microneedle modules via a digitally-adjustable momentum is preferably above 5 cm/second.

In an embodiment of this invention, the application momentum that is obtained by a solenoid or a voice coil is controlled by adjusting the actuator power.

Changing the position of a supporting plateau in a variable time interval from a retracted position towards a zero position by changing the duty cycle of a pulse width modulation, results in propelling the supporting plateau with a certain velocity towards the zero-position.

In a context of this invention, the “actuator distance” is the distance between the position of a supporting plateau that is mounted onto an actuator in a non-extended position and in a fully extended position. This distance for a specific supporting plateau mounted actuator may be predetermined by using a ruler. The “actuator distance” may also be determined by using a position sensor, such as a linear encoder that is coupled to the inner housing and a movable part of the actuator that moves in the same extent as the supporting plateau, which may be used to continuously determine the position of a supporting plateau.

In another context of this invention, the “activation time” is the time between the actuator is digitally-powered and the time that the actuator is in its maximum extended position, which is digitally measured by the controller.

In an embodiment of the invention, the maximum extended position of the actuator-supporting plateau combination is determined by measuring a signal from a piezoelectric element 125 that is inside the applicator outer shell 101, preferably mounted onto the inner housing 107, see FIG. 2, that will exceed a threshold voltage when the actuator reaches its maximum extended position.

In another embodiment of the invention, the average application velocity is calculated by the controller by dividing the actuator distance by the activation time.

In an embodiment of this invention, a microneedle module is applied onto skin by a digitally-controlled momentum, as schematically represented in FIG. 5A-5C. A microneedle module is mounted onto a supporting plateau, which is in a retracted position. The applicator's skin support is placed perpendicularly onto skin. Upon increasing the actuator power, the supporting plateau onto which a microneedle module is mounted, propels towards a zero-position. This results in application of a microneedle module with a certain velocity onto skin where it is retained until a digitally-installed application time is reached, upon which the supporting plateau, including the microneedle module, moves to a retracted position.

In another embodiment of the invention, the microneedle module is applied onto skin by a digitally controlled momentum, as schematically represented in FIGS. 5D-5F. A microneedle module is placed and/or mounted onto skin. The applicator's skin support is placed perpendicularly onto skin. Upon increasing the actuator power, the supporting plateau propels towards a zero-position towards the skin and microneedle module. The supporting plateau is retained in a zero-position until a digitally-installed application time is reached. Finally, the supporting plateau moves to a retracted position, leaving the microneedle module applied onto the skin.

Applying a microneedle module onto skin for a prolonged time may result in heat production by the solenoid. To reduce heat production in the solenoid, the actuator power may be reduced to a minimum while still able to retain a supporting plateau in a zero-position. Reducing the actuator power may be established by adjusting the duty cycle of the PWM signal 520, see also FIG. 6. Preferably the actuator power is reduced to a minimum after the supporting plateau is in a zero position.

Optionally, the temperature of the actuator in the applicator is monitored by the controller through a temperature sensor that is attached onto or in close proximity of the actuator. When the temperature exceeds a threshold value, the controller may automatically remove the actuator power to prevent overheating of the actuator and/or prevent temperature discomfort when holding the applicator and/or using the applicator to apply a microneedle module onto skin.

An applicator according to an embodiment is shown in FIG. 2. This applicator comprises an outer housing that may be hold by hand to apply a microneedle module onto the skin. The outer housing may comprise an applicator outer shell 101, a top lid 102 and a skin support 100. The top lid 102 of the applicator 4 contains a connector 103 that is used to connect the applicator 4 to a controller 3 arranged to control the manner of application and the insertion parameters.

Furthermore, on the top lid 102 there is a button 104, of which its state is read by the controller, to activate the applicator when applying a microneedle module onto the skin. On the top lid there is an adjustment bolt 105 that is used to move an inner housing 107 via a screw thread 112 in the displacement cap 113 of the inner housing vertically (i.e. in a direction along a main axis of the actuator), up and down within the outer housing. By turning the adjustment bold the inner housing 107 vertically moves up or down. In this example, the inner housing 107 is kept in place by a spring 106 around the adjustment bolt 105. Further, rubber rings 108 are used between an inner housing 107 and outer applicator shell 101 to vertically align the inner housing 107. Furthermore, the applicator 4 comprises a limiting bolt 109 on the outer applicator shell and limiting nut 110 that is guided through a limiting notch 111 to restrain the movement of the inner housing within the outer applicator shell over a certain distance, e.g. 2 cm. The actuator 114 in the applicator 4 may be a solenoid 114. FIG. 2 shows a rod 116 movable through the solenoid 114. Onto a first outer end of the rod 116 a supporting plateau 115 is mounted that may be aligned with an opening 99 in a skin support 100 to apply a dermal microneedle module (not shown) onto skin.

On an opposite end of the rod 116 an end stop 118 is mounted and a bias spring 117 is arranged to produce a counter force when the rod 116 is moved due to activation of the actuator 114. This actuator 114 may be a solenoid or a voice coil arranged to force the supporting plateau 115 out of the outer housing 100,101,102. If the actuator 114 is not activated, or not sufficiently activated, the bias spring 117 will urge the rod 116, and thus the supporting plateau 115 back towards a retracted position.

In an embodiment, the supporting plateau 115 is adjustable into three different positions as shown in FIGS. 3A-3C which show three adjustable positions of the supporting plateau of the applicator 4 with a microneedle module attached.

The supporting plateau 115 and skin support 100 are exchangeable to fit microneedle modules dependent on their size, whereby the diameter of the skin support is smaller than the diameter of the opening 99 in the skin support 100.

The applicator 4 that comprises an actuator 114 onto which the supporting plateau 115 is mounted, onto which a microneedle module 120 is attached may be in a retracted position, see FIG. 3A, a zero position, see FIG. 3B and a protruding position, see FIG. 3C, in relation to a skin support 100. By another method of application via pressure, a microneedle module may be placed and/or attached onto the skin, resulting in a possible other zero position of the supporting plateau as compared to a mounted microneedle module. In this case, a supporting plateau without a mounted microneedle module may also be in a retracted position, see FIG. 3D, a zero position, see FIG. 3E, and a protruding position, see FIG. 3F, in relation to a skin support.

When the actuator in the applicator 4 is a solenoid or a voice coil, the position of the supporting plateau 115 may be adjusted to a zero-position or a protruding position when the actuator is fully extended by using an adjustment bolt as represented in FIG. 2. The supporting plateau 115 is in a retracted position when the actuator is not fully extended. However, these positions may also be digitally adjustable by changing the duty cycle of a pulse width modulation.

Optionally, the actuator in the applicator may be a solenoid, voice coil, or a stepper motor, wherein the position of the supporting plateau is monitored through a position sensor (such as a linear encoder). The position of a supporting plateau is digitally set to a zero position or a protruding position via the controller through feedback by a position sensor.

The system 1 can be used to apply a microneedle module 120 onto skin 300 by applying a digitally-adjustable pressure, as represented in FIG. 4. The actuator 114 may be a solenoid or a voice coil. By this manner of application, a microneedle module may be mounted onto a supporting plateau 115 that is mounted onto an actuator, shown in FIGS. 4A-4C. Initially, a supporting plateau is in a protruded position, see FIG. 4A. Next, the supporting plateau onto which a microneedle module is mounted is pressed perpendicularly onto the skin, see FIG. 4B. When a digitally-installed pressure is obtained, the supporting plateau partially retracts towards the applicator into a zero position, where it is retained until a digitally-installed application time is reached, upon which the supporting plateau, including the microneedle module, moves into a retracted position, see FIG. 4C.

As an alternative to apply a microneedle onto skin by a digitally-adjustable pressure, a microneedle module is placed and/or mounted onto skin as shown in FIG. 4D-4F. The actuator may be a solenoid or a voice coil. First, the supporting plateau is in a protruding position, see FIG. 4D, and is subsequently pressed perpendicularly onto the microneedle module, see FIG. 4E. When the digitally installed pressure is obtained, the supporting plateau partially retracts towards the applicator into a zero-position, where it is retained until a digitally-installed application time is reached. Finally, the supporting plateau moves to a retracted position, leaving the microneedle module applied onto the skin, see FIG. 4F.

Optionally, the actuator in an applicator may be a stepper motor or a voice coil, wherein the application pressure is digitally adjusted and regulated via feedback of a pressure sensor. Hereby is a skin support 100 placed perpendicularly onto the skin, wherein a microneedle module is between a supporting plateau and the skin. To apply a microneedle module a supporting plateau moves towards the skin until the digitally installed pressure is obtained. This pressure will be retained until a digitally installed application time is reached upon which the supporting plateau retracts into the applicator.

The system as disclosed in this invention can be used to apply a microneedle module 120 onto skin 300 by applying a digitally-adjustable momentum, as represented in FIG. 5. The actuator 114 may be a solenoid or a voice coil. By this manner of application, a microneedle module may be mounted onto a supporting plateau 115 that is mounted onto an actuator, shown in FIG. 5A-5C. Initially, the supporting plateau is in a retracted position, see FIG. 5A. The applicator's skin support 100 is placed perpendicularly onto skin. Upon increasing the duty cycle of a pulse width modulation, the supporting plateau onto which a microneedle module is mounted, propels towards a zero position, see FIG. 5B. This results in application of a microneedle module with a certain velocity onto skin where it is retained until a digitally-installed application time is reached, upon which the supporting plateau, including the microneedle module, moves to a retracted position, see FIG. 5C.

As an alternative to apply a microneedle onto skin by a digitally adjustable momentum, a microneedle module is placed and/or mounted onto skin, as shown in FIG. 5D-5F. The applicator's skin support is placed perpendicularly onto skin, see FIG. 5D. Upon increasing the duty cycle of a pulse width modulation, the supporting plateau propels towards a zero position towards the skin and microneedle module, see FIG. 5E. The supporting plateau 115 is retained in a zero position until a digitally-installed application time is reached. Finally, the supporting plateau 115 moves to a retracted position, leaving the microneedle module applied onto the skin, see FIG. 5F.

In an embodiment the electronic controller 3 is arranged to digitally adjust the manner of application and application parameters by which the applicator applies a microneedle module onto skin, wherein the actuator may be controlled via controlling a pulse width modulation (PWM). As shown in FIG. 6, the electronic controller 3 contains an integrated circuit (IC) 500 to digitally generate a PWM signal 520 at a PWM frequency of at least 1 kHz, but preferably above 10 kHz. The duty cycle of the PWM signal 520 is used to control the movement and/or position of the supporting plateau 115. With no PWM signal applied to the actuator, the supporting plateau 115 is in the retracted position, with a sufficient width in the PWM signal, the supporting plateau 115 is in the protruded position.

A diode 504 in series with a resistor 510 is used to protect the IC 500 against a potential backward voltage by the MOSFET 507. A NPN transistor 509 switches the ground 503 at the PWM frequency. The circuit contains a pull-down resistor 511 between the base of the NPN transistor 509 and the ground 503 and a pull-up resistor 512 between the emitter of the NPN transistor 509 and positive DC voltage 501. Subsequently, the PWM signal arriving from the collector of the NPN arrives at the base of a PNP transistor 508 via a resistor 513 to switch positive DC voltage 501. A voltage divider, comprising a resistor 514 and a variable resistor 515, is used to regulate the switching voltage on the gate of the MOSFET 507. The MOSFET 507 is arranged to control the current through the actuator 506 so as to switch the actuator 506 on and off. The actuator 506 is connected to a positive DC voltage 502 and paralleled with a fly back diode 505. It is noted the FIG. 6 shows an exemplary embodiment and that other switching circuitries could be designed which are arranged to switch the actuator 506 on and off the switching circuitry being controlled by a PWM signal.

### Example 1

A controller according to FIG. 1 and an applicator according to FIG. 2 were used in a mode to deliver a certain amount of force via a supporting plateau (as shown in FIG. 4). Three different push-type solenoids were used in three different applicators. The pressure of the three actuators as a function of the actuator power was investigated by adjusting the actuator power and measuring the maximum holding force of the solenoid, with the actuator in a protruded position.

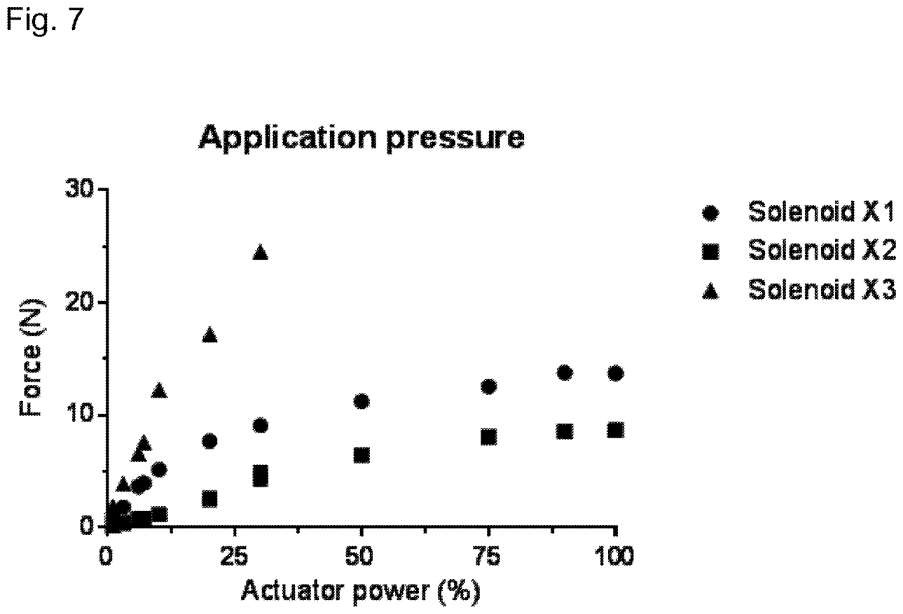

As shown in FIG. 7, the pressure of the actuators was controlled by digitally adjusting the actuator power on the controller. By using solenoid X1 the pressure was controlled between 0.95 Newton (1%) and 13.8 Newton (100%) by digitally adjusting the actuator power on the controller. By using solenoid X2 the pressure was controlled between 0.20 Newton (1%) and 8.7 Newton (100%) by digitally adjusting the actuator power on the controller. By using solenoid X3 the pressure was controlled between 1.9 Newton (1%) and 24.5 Newton (30%) by digitally adjusting the actuator power on the controller. Increasing the actuator power for solenoid X3 above 30% resulted in a further increased force, however, the available equipment did not allow us to accurately determine this force.

### Example 2

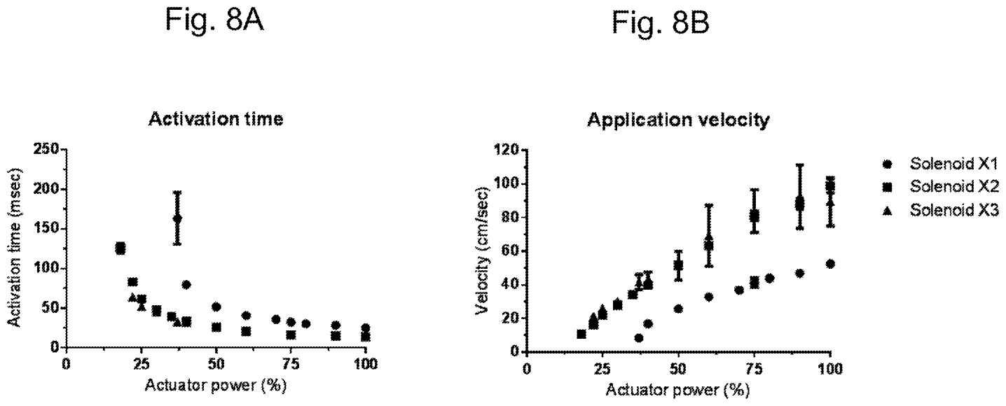

A controller according to FIG. 1 and an applicator according to FIG. 2 were used in a mode to deliver a certain momentum via a supporting plateau (as shown in FIG. 5). Three different push-type solenoids were used in three different applicators. The activation time to move the supporting plateau from a retracted position into a zero-position of the three actuators as a function of the actuator power was measured by the controller, as shown in FIG. 8. The actuator distance between the retracted position and the zero-position was measured by using a digital ruler (Solenoid X1: 13.3 mm; Solenoid X2: 8.55 mm; Solenoid X3: 13.5 mm). Subsequently, the average velocity was calculated by dividing the actuator distance by the activation time.

As shown in FIG. 8, the momentum of the supporting plateau via the actuators was controlled by digitally adjusting the actuator power on the controller. By using solenoid X1 the average velocity was controlled between 8.3 cm/second (37%) and 52.5 cm/second (100%) by digitally adjusting the actuator power on the controller. By using solenoid X2 the average velocity was controlled between 10.7 cm/second (18%) and 98.9 cm/second (100%) by digitally adjusting the actuator power on the controller. By using solenoid X3 the average velocity was controlled between 21.1 cm/second (22%) and 89.6 cm/second (100%) by digitally adjusting the actuator power on the controller.

An applicator-controller combination as described in the current invention is particularly suitable for researchers, companies and research institutes that develop microneedles to investigate and determine the optimal application parameters of microneedle modules onto skin but may not be restricted to it.

It should be noted that the above-mentioned embodiments illustrate rather than limit the invention, and that those skilled in the art will be able to design many alternative embodiments.

In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. In the device claim enumerating several means, several of these means may be embodied by one and the same item of hardware or software. The mere fact that certain measures are recited in mutually different dependent claims does not indicate that a combination of these measures cannot be used to advantage.

1. A microneedle applicator system, the system comprising:
an applicator comprising an outer housing and an electromechanical actuator arranged in the outer housing and arranged to apply a microneedle module via a supporting body onto skin, the electromechanical actuator being arranged to displace the supporting body between a first position in which the supporting body is positioned inside the housing, and a second position in which the supporting body is substantially in line with an opening in the housing or outside the housing;
a controller arranged to control a velocity of the actuator and/or a pressure force of the actuator by regulating the power that is supplied to the actuator, wherein the controller comprises:
a switching circuitry for switching the power supplied to the actuator on and off, and
an integrated circuit arranged to control the switching circuitry.

2. The microneedle applicator system according to claim 1, wherein the actuator comprises a solenoid or a voice coil arranged to move the supporting body from a first position to a second position within or outside the applicator.

3. (canceled)

4. The microneedle applicator system according to claim 1, wherein the applicator comprises an inner housing adjustably arranged inside the outer housing wherein by adjusting the position of the inner housing along a main axis of the actuator, the second position of the supporting body can be set between a zero-position in which the supporting body is substantially in line with the opening and a protruded position in which the supporting body is outside the outer housing.

5. The microneedle applicator system according to claim 4, wherein the applicator comprises a piezoelectric element arranged onto the inner housing, wherein the controller is arranged to determine a maximum extended position of the supporting body by measuring a signal from the piezoelectric element and comparing it to a threshold voltage, and if the measured signal exceeds the threshold voltage, retract the supporting body into the outer housing after a predetermined period of time.

6. The microneedle applicator system according to claim 1, wherein the supporting body is adjustably arranged onto the actuator wherein by adjusting the position of the supporting body along a main axis of the actuator, the second position of the supporting body can be set between a zero-position in which the supporting body is substantially in line with the opening and a protruded position in which the supporting body is outside the outer housing.

7. The microneedle applicator system according to claim 1, wherein the actuator is controlled in such a way that:
I. at first, the position of the actuator is set in such a way that the supporting body protrudes out of the applicator;
II. subsequently, after the applicator is applied to the skin by a user pressing the microneedle module via the supporting body onto skin the supporting body is partially protruded when the holding force is reached, the supporting body remains partially protruded for a predetermined period of time, for holding the microneedle module onto skin for the predetermined period of time; and
III. subsequently, the supporting body is retracted into the applicator when a predetermined time is reached.

8. The microneedle applicator system according to claim 1, wherein the actuator is controlled in such a way that:
I. at first, the position of the actuator is set in such a way that the supporting body is inside the applicator;
II. subsequently, after the applicator is placed onto skin by a user, the actuator moves the supporting body towards the skin until the supporting body reaches the skin, wherein the supporting body is pressing the microneedle module onto skin until a predetermined pressure is reached;
III. subsequently, the supporting body remains at its position holding the microneedle module onto skin for a predetermined period of time; and
IV. subsequently, the supporting body is retracted into the applicator.

9. The microneedle applicator system according to claim 1, wherein the actuator is controlled in such a way that:
I. at first, the supporting body is in a retracted state in the applicator;
II. subsequently, after the applicator is placed onto the skin by a user, the supporting body is propelled towards the second position;
III. subsequently, the supporting body is retained in the second position for a predetermined period of time; and
IV. subsequently, the supporting body is retracted into the applicator.

10. The microneedle applicator system according to claim 9, wherein the supporting body is propelled multiple times with a predetermined application frequency towards the second position.

11. The microneedle applicator system according to claim 10, wherein the application frequency is adjustable between 0.01 mHz and 50 Hz.

12. The microneedle applicator system according to claim 11, wherein the controller (3) is arranged to adjust the number of insertions between 2 and 10\hat{ }6.

13. The microneedle applicator system according to claim 10, wherein the controller is arranged to adjust a time and/or ratio that the supporting body is in the second position and (partially) retracted in the outer housing of the applicator within one insertion cycle, wherein one insertion cycle is the time that the actuator is in the second position plus the time that the actuator is (partially) retracted into the outer housing of the applicator.

14. The microneedle applicator system according to claim 1, wherein the controller is arranged to read a value indicative of an amount of pressure force onto the supporting body or indicative of a momentum of the actuator from a calibration table.

15. The microneedle applicator system according to claim 1, wherein the system comprises a pressure sensor, at least in use between the skin and the actuator, and arranged to sense an applied pressure on the supporting body, wherein the controller is arranged to control the power of the applicator using feedback from the pressure sensor.

16. The microneedle applicator system according to claim 1, wherein the system comprises a sensor arranged to measure a velocity of the actuator, wherein the controller is arranged to calculate or receive the measured velocity and to control an average velocity of the actuator between a retracted position and the second position or the velocity at the second position using feedback of the velocity sensor.

17. The microneedle applicator system according to claim 16, wherein the sensor is a linear encoder or a piezoelectric element.

18. The microneedle applicator system according to claim 1, wherein the controller is arranged to execute a software program so as to digitally adjust the time that the supporting body is in the first and/or second position and/or delivers a required force to retain a drug delivery and/or sampling device for a pre-installed application time onto the skin.

19.-23. (canceled)

24. The microneedle applicator system according to claim 1, wherein the integrated circuit is arranged to generate a modulated digital signal to control the switching circuitry.

25. The microneedle applicator system according to claim 24, wherein the modulated digital signal is a pulse width modulated signal.

26. The microneedle applicator system according to claim 25, wherein the integrated circuit controls the integrated circuit by setting or adjusting a duty cycle of the pulse width modulated signal.
